(19) United States
(12) Patent Application Publication
(10) Pub. No.: US 2026/0274850 A1
(43) Pub. Date: Sep. 17, 2026

(54) SALTS AND SOLID FORMS OF ELENESTINIB

(71) Applicant: ASSIA CHEMICAL INDUSTRIES LTD., Tel Aviv (IL)

(72) Inventors: Valentina Travancic, Zagreb (HR); Dijana Škalec Šamec, Jastrebarsko (HR)

(21) Appl. No.: 19/157,005

(22) PCT Filed: Feb. 16, 2024
(86) PCT No.: PCT/IB2024/051505

§ 371 Date: Aug. 15, 2025

Related U.S. Application Data
(60) Provisional application No. 63/446,427, filed on Feb. 17, 2023.
(60) Provisional application No. 63/453,523, filed on Mar. 21, 2023.
(60) Provisional application No. 63/627,888, filed on Feb. 1, 2024.

(51) Int. Cl. C07D 487/04; A61K 31/53; C07C 55/10; C07C 57/15; C07C 309/19

(52) U.S. Cl. CPC C07D 487/04; A61K 31/53; C07C 55/10; C07C 57/15; C07C 309/19; C07B 2200/13

(57) ABSTRACT

The present disclosure encompasses salts and solid-state forms of Elenestinib, processes for preparation thereof and pharmaceutical compositions thereof.

### FIELD OF THE DISCLOSURE

The present disclosure encompasses salts and solid-state forms of Elenestinib, processes for preparation thereof and pharmaceutical compositions thereof.

### BACKGROUND OF THE DISCLOSURE

Elenestinib, also known as BLU-263, has the chemical name: (S)-2-(4-(4-(4-(5-(1-amino-1-(4-fluorophenyl)ethyl)pyrimidin-2-yl)piperazin-1-yl)pyrrolo[2,1-f][1,2,4]triazin-6-yl)-1H-pyrazol-1-yl)ethan-1, and the following chemical structure:

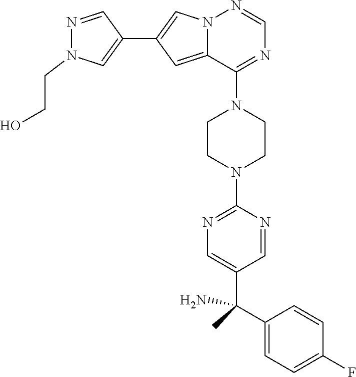 [SMILES: C[C@](N)(c1ccc(F)cc1)c1cnc(N2CCN(c3ncnn4cc(-c5cnn(CCO)c5)cc34)CC2)nc1]

Elenestinib is developed for the treatment of disorders and conditions associated with Oncogenic KIT and PDGFRA alterations such as systemic mastocytosis gastrointestinal stromal tumors or acute myeloid leukemia.

Elenestinib, referred herein also as “Compound (I)” is described in U.S. Pat. No. 10,829,493. Crystalline forms and Specific salts of Compound (I) (Phosphate, Besylate, Benzoate and Sulfate) are described in International Publication No. WO2022192558.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new salt, solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability).

More specifically, WO2022192558 discloses that the preparation of a stable crystalline form of the free base of Elenestinib was found to be difficult to reproduce on a production scale. The crystalline form A of Elenestinib phosphate as described in WO2022192558 was not stable under strong and solvent drop grinding and showed conversion to amorphous material. Further, also the preparation of crystalline Form A of Elenestinib phosphate was difficult and often a gum was obtained.

For at least these reasons, there is a need for additional salts and solid state forms of Elenestinib.

### SUMMARY OF THE DISCLOSURE

The present disclosure provides salts and co-crystals of Elenestinib and crystalline forms thereof processes for their preparation and pharmaceutical compositions thereof. Any one of the crystalline forms of the present disclosure can be used to prepare other solid-state forms of Elenestinib or salts thereof

The present disclosure also provides uses of said salts and crystalline forms of Elenestinib in the preparation of other solid state forms of Elenestinib or other salts and their solid state forms thereof.

The present disclosure provides said salts and crystalline forms thereof for use in medicine, including for the treatment of systemic mastocytosis gastrointestinal stromal tumors or acute myeloid leukemia.

The present disclosure also encompasses the use of salts and crystalline polymorphs of the present disclosure for the preparation of pharmaceutical compositions and/or formulations.

In another aspect, the present disclosure provides pharmaceutical compositions comprising salts and crystalline polymorphs of Elenestinib according to the present disclosure. Pharmaceutical compositions according to any aspect of the present disclosure may include oral dosage forms.

The present disclosure includes processes for preparing the above mentioned pharmaceutical compositions. The processes include combining any one or a combination of the salts and crystalline polymorphs of Elenestinib with at least one pharmaceutically acceptable excipient.

The salts and crystalline polymorphs of Elenestinib as defined herein and the pharmaceutical compositions or formulations comprising them may be used as medicaments, such as for the treatment of patients suffering from systemic mastocytosis gastrointestinal stromal tumors or acute myeloid leukemia.

The present disclosure also provides methods of treating disorders and conditions associated with Oncogenic KIT and PDGFRA alterations by administering a therapeutically effective amount of any one or a combination of the salts and crystalline polymorphs of Elenestinib of the present disclosure, or at least one of the above pharmaceutical compositions, to a subject suffering from systemic mastocytosis or otherwise in need of the treatment

The present disclosure also provides uses of salts and crystalline polymorphs of Elenestinib of the present disclosure, or at least one of the above pharmaceutical compositions, for the manufacture of medicaments for treating e.g. systemic mastocytosis

### BRIEF DESCRIPTION OF THE DRAWINGS

FIG. 1 shows the X-ray powder diffraction pattern (XRPD) of Elenestinib Camsylate Form C1.

FIG. 2 shows the X-ray powder diffraction pattern (XRPD) of Elenestinib Succinate form S1.

FIG. 3 shows X-ray powder diffraction pattern (XRPD) of Elenestinib Nicotinate form N1.

FIG. 4 shows X-ray powder diffraction pattern (XRPD) of Elenestinib:Fumaric acid form F1.

FIG. 5 shows X-ray powder diffraction pattern (XRPD) of Elenestinib:L-lactic acid form L1.

FIG. 6 shows X-ray powder diffraction pattern (XRPD) of Elenestinib:Gluconic acid form G1.

FIG. 7 shows X-ray powder diffraction pattern (XRPD) of Elenestinib Camsylate Form C2.

FIG. 8 shows X-ray powder diffraction pattern (XRPD) of Elenestinib Phosphate Form P1.

FIG. 9 shows a Differential Scanning Calorimetry (DSC) thermogram of Elenestinib Camsylate Form C1.

FIG. 10 shows a DSC thermogram of Elenestinib Camsylate Form C2.

### DETAILED DESCRIPTION OF THE DISCLOSURE

The present disclosure encompasses Elenestinib salts, co-crystals and crystalline polymorphs thereof, processes for preparation thereof, and pharmaceutical compositions thereof.

Solid state properties of Elenestinib salts and crystalline polymorphs thereof can be influenced by controlling the conditions under which Elenestinib and crystalline polymorphs thereof are obtained in solid form.

The solid state forms of Elenestinib salts or co-crystals as described in any aspect or embodiment of the present disclosure may be polymorphically pure, or substantially free of any other solid state (or polymorphic) forms.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. For example, polymorphically pure Elenestinib form C1 means that the solid state form is substantially free of other solid state forms of Elenestinib. Thus, a crystalline polymorph of Elenestinib salts described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Elenestinib salts. In some embodiments of the disclosure, the described crystalline polymorph of Elenestinib salt or cocrystal may contain from about 1% to about 20% (w/w), from about 5% to about 20% (w/w), or from about 5% to about 10% (w/w) of one or more other crystalline polymorph of the same Elenestinib.

Depending on which other crystalline polymorphs a comparison is made, the crystalline polymorphs of Elenestinib salts or co-crystals of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density.

The solid state forms of Elenestinib of the present disclosure are stable under different stress conditions like heating, pressure, different grinding conditions and to humidity.

Further, the solid state forms of Elenestinib of the present disclosure can easily and reproducibly obtained by crystallization experiments.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Elenestinib referred to herein as being characterized by graphical data “as depicted in” or “as substantially depicted in” a Figure will thus be understood to include any crystal forms of Elenestinib characterized with the graphical data having such small variations, as are well known to the skilled person, in comparison with the Figure.

As used herein, and unless stated otherwise, the term “anhydrous” in relation to crystalline forms of Elenestinib salts or co-crystals relates to a crystalline form of Elenestinib which does not include any crystalline water (or other solvents) in a defined, stoichiometric amount within the crystal. Moreover, an “anhydrous” form would generally not contain more than 1% (w/w) of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

“Co-Crystal” or “Co-crystal” as used herein is defined as a crystalline material including two or more molecules in the same crystalline lattice and associated by non-ionic and non-covalent bonds. In some embodiments, the co-crystal includes two molecules which are in natural state. In an embodiments the molar ratio between the active pharmaceutical ingredient (Elenestinib) and the coformer (for example: Fumaric acid) is between 2:1 and 1:2, or between 1.5:1 and 1:1.5, preferably between 1.25:1 and 1:1.25, in other embodiments about 1:1.

As used herein, the term “isolated” in reference to crystalline polymorph of Elenestinib of the present disclosure corresponds to a crystalline polymorph of Elenestinib that is physically separated from the reaction mixture in which it is formed.

As used herein, unless stated otherwise, the XRPD measurements are taken using copper K α radiation wavelength 1.54184 Å. XRPD peaks reported herein are optionally measured using CuK α radiation, λ=1.54184 Å, typically at a temperature of 25±3° C.

A thing, e.g., a reaction mixture, may be characterized herein as being at, or allowed to come to “room temperature” or “ambient temperature,” often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

The solid state form of Elenestinib salt or co crystal as described in any aspect or embodiment of the present disclosure may be chemically pure, or substantially free of any other compounds.

A compound may be referred to herein as chemically pure or purified compound or as substantially free of any other compounds. As used herein, the terms “chemically pure” or “purified” or “substantially free of any other compounds” refer to a compound that is substantially free of any impurities including enantiomers of the subject compound, A chemically pure or purified compound or a compound that is substantially free of any other compound will be understood to mean that it contains about 10% (w/w) or less, about 5% (w/w) or less, about 4% (w/w) or less, about 3% (w/w) or less, about 2% (w/w) or less, about 1.5% (w/w) or less, about 1% (w/w) or less, about 0.8% (w/w) or less, about 0.6% (w/w) or less, about 0.4% (w/w) or less, about 0.2% (w/w) or less, about 0.1% (w/w) or less, or about 0% of any other compound as measured, for example, by HPLC. Thus, pure or purified Elenestinib salt or Co-crystals described herein as substantially free of any compounds would be understood to contain greater than about 90% (w/w), greater than about 95% (w/w), greater than about 96% (w/w), greater than about 97% (w/w), greater than about 98% (w/w), greater than about 98.5% (w/w), greater than about 99% (w/w), greater than about 99.2% (w/w), greater than about 99.4% (w/w), greater than about 99.6% (w/w), greater than about 99.8% (w/w), greater than about 99.9% (w/w), or about 100% of the subject Elenestinib salt or co-crystals

As used herein, the salt of the present disclosure “Elenestinib Camsylate” refers to a salt of Elenestinib and camphorsulfonic acid, wherein the molar ratio between Elenestinib and camphorsulfonic acid is about 1:1 or about 2:1 or about 1:2.

As used herein, the salt of the present disclosure “Elenestinib Succinate” refers to a salt of Elenestinib and succinic acid, wherein the molar ratio between Elenestinib and succinic acid is about 1:1 or about 2:1 or about 1:2.

As used herein, the salt of the present disclosure “Elenestinib Nicotinate” refers to a salt of Elenestinib and nicotinic acid, wherein the molar ratio between Elenestinib and nicotinic acid is about 1:1 or about 2:1 or about 1:2

In one aspect, the present disclosure provides Elenestinib Camsylate. In certain embodiments, Elenestinib Camsylate has a molar ratio between Elenestinib and the Camphorsulfonic acid 1:1. In preferred embodiments, Elenestinib Camsylate is in crystalline form.

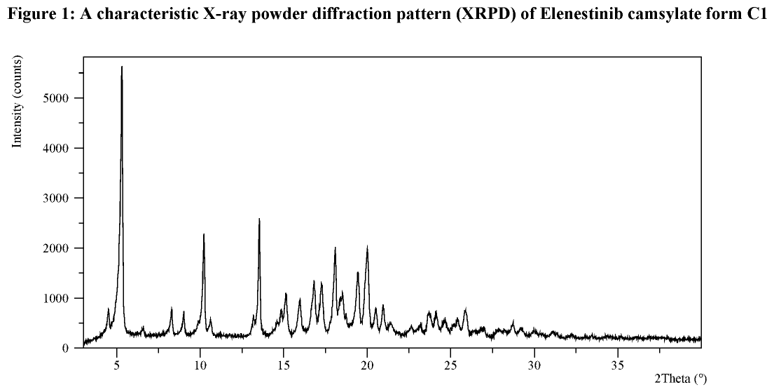

The present disclosure includes a crystalline polymorph Elenestinib Camsylate designated Form C1. The crystalline Form C1 of Elenestinib Camsylate may be characterized by data selected from one or more of the following: an X-ray powder diffraction pattern substantially as depicted in FIG. 1; an X-ray powder diffraction pattern having peaks at 5.3, 10.2, 13.6, 18.1 and 20.0 degrees 2-theta±0.2 degrees 2-theta; and combinations of these data.

Crystalline Form C1 of Elenestinib Camsylate may be further characterized by an X-ray powder diffraction pattern having peaks at 5.3, 10.2, 13.6, 18.1 and 20.0 degrees 2-theta±0.2 degrees 2-theta, and also having any one, two, three, four or five additional peaks selected from 8.3, 16.0, 16.8, 17.3 and 19.4 degrees 2-theta±0.2 degrees 2-theta.

Crystalline Form C1 of Elenestinib Camsylate may be alternatively characterized by an X-ray powder diffraction pattern having peaks at 5.3, 8.3, 10.2, 13.6, 16.0, 16.8, 17.3, 18.1, 19.4 and 20.0 degrees 2-theta±0.2 degrees 2-theta

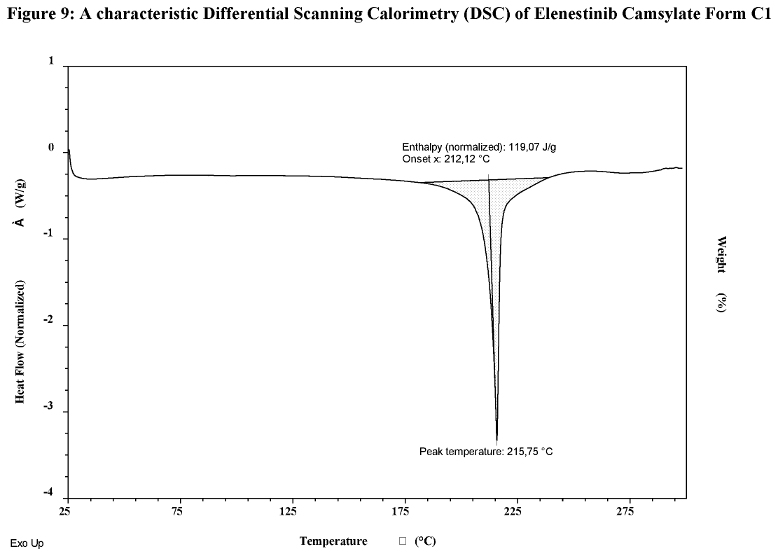

Crystalline Form C1 of Elenestinib Camsylate may be further characterized by an endothermic peak at 215.75 degrees Celsius in DSC thermogram. Crystalline Form C1 of Elenestinib Camsylate may be further characterized by DSC thermogram substantially as depicted in FIG. 9.

In any aspect or embodiment of the present disclosure, crystalline Form C1 of Elenestinib Camsylate may be isolated.

In any aspect or embodiment crystalline form C1 of Elenestinib Camsylate may be chemically pure.

In any aspect or embodiment crystalline Form C1 of Elenestinib Camsylate may be polymorphically pure.

Crystalline Form C1 of Elenestinib Camsylate may be characterized by each of the above characteristics alone/or by all possible combinations.

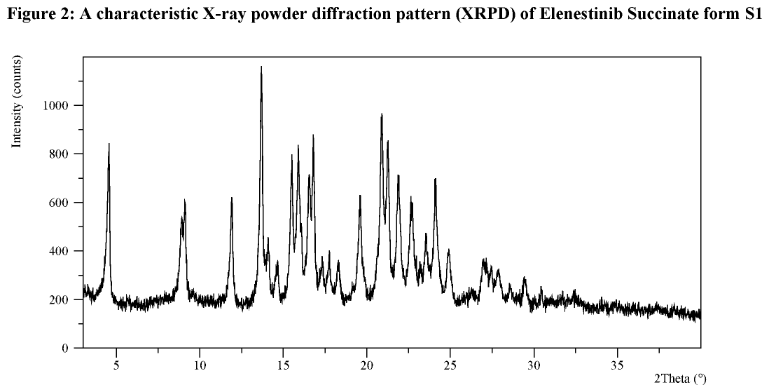

In another aspect, the present disclosure provides Elenestinib Succinate. In certain embodiments, Elenestinib Succinate has a molar ratio between Elenestinib and the Succinic acid 1:1. In preferred embodiments, Elenestinib Succinate is in crystalline form. The present disclosure includes a crystalline polymorph Elenestinib Succinate designated Form S1. The crystalline Form S1 of Elenestinib Succinate may be characterized by data selected from one or more of the following: an X-ray powder diffraction pattern substantially as depicted in FIG. 2; an X-ray powder diffraction pattern having peaks at 4.6, 11.9, 13.7, 19.6 and 20.9 degrees 2-theta±0.2 degrees 2-theta; and combinations of these data.

Crystalline Form S1 of Elenestinib Succinate may be further characterized by an X-ray powder diffraction pattern having peaks at 4.6, 11.9, 13.7, 19.6 and 20.9 degrees 2-theta±0.2 degrees 2-theta, and also having any one, two, three, four or five additional peaks selected from 15.5, 16.8, 17.7, 21.9 and 24.1 degrees 2-theta±0.2 degrees 2-theta.

Crystalline Form S1 of Elenestinib Succinate may be alternatively characterized by an X-ray powder diffraction pattern having peaks at 4.6, 11.9, 13.7, 15.5, 16.8, 17.7, 19.6, 20.9, 21.9 and 24.1 degrees 2-theta±0.2 degrees 2-theta.

In any aspect or embodiment of the present disclosure, crystalline Form S1 of Elenestinib Succinate may be isolated.

In any aspect or embodiment crystalline form S1 of Elenestinib Succinate may be chemically pure.

In any aspect or embodiment crystalline Form S1 of Elenestinib Succinate may be polymorphically pure.

Crystalline Form S1 of Elenestinib Succinate may be characterized by each of the above characteristics alone/or by all possible combinations.

In another aspect, the present disclosure provides Elenestinib Nicotinate. In certain embodiments, Elenestinib Nicotinate has a molar ratio between Elenestinib and the nicotinic acid 1:1. In preferred embodiments, Elenestinib Nicotinate is in crystalline form.

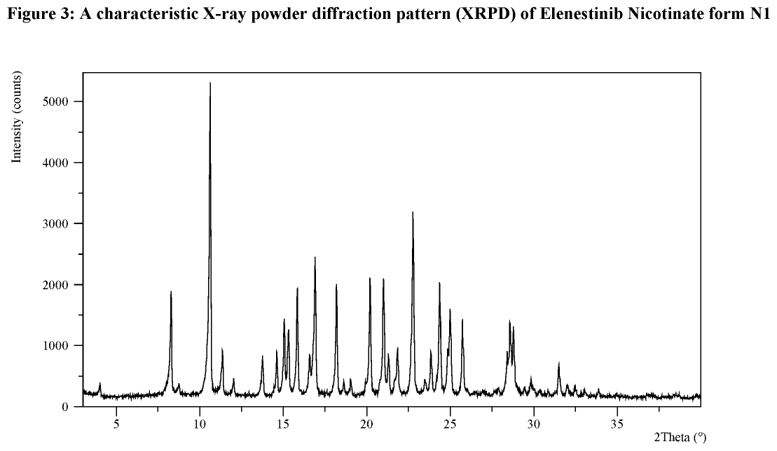

The present disclosure includes a crystalline polymorph Elenestinib Nicotinate designated Form N1. The crystalline Form N1 of Elenestinib Nicotinate may be characterized by data selected from one or more of the following: an X-ray powder diffraction pattern substantially as depicted in FIG. 3; an X-ray powder diffraction pattern having peaks at 8.3, 10.6, 16.9, 20.2 and 22.8 degrees 2-theta±0.2 degrees 2-theta; and combinations of these data.

Crystalline Form N1 of Elenestinib Nicotinate may be further characterized by an X-ray powder diffraction pattern having peaks at 8.3, 10.6, 16.9, 20.2 and 22.8 degrees 2-theta 0.2 degrees 2-theta, and also having any one, two, three, four or five additional peaks selected from 15.8, 18.2, 21.0, 24.4 and 25.0 degrees 2-theta±0.2 degrees 2-theta.

Crystalline Form N1 of Elenestinib Nicotinate may be alternatively characterized by an X-ray powder diffraction pattern having peaks at 8.3, 10.6, 15.8, 16.9, 18.2, 20.2, 21.0, 22.8, 24.4 and 25.0 degrees 2-theta±0.2 degrees 2-theta.

In any aspect or embodiment of the present disclosure, crystalline Form N1 of Elenestinib Nicotinate may be isolated.

In any aspect or embodiment crystalline form N1 of Elenestinib Nicotinate may be chemically pure.

In any aspect or embodiment crystalline Form N1 of Elenestinib Nicotinate may be polymorphically pure.

Crystalline Form N1 of Elenestinib Nicotinate may be characterized by each of the above characteristics alone/or by all possible combinations

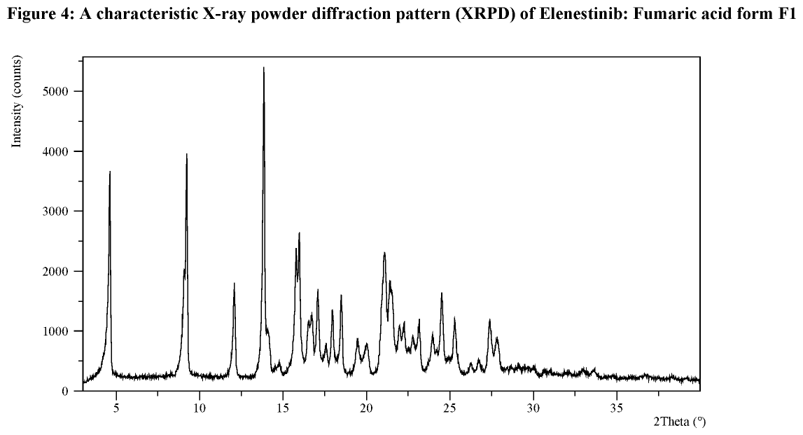

The disclosure further encompasses a crystalline form of Elenestinib:Fumaric acid, designated form F1. Crystalline Form F1 of Elenestinib:Fumaric acid may be characterized by data selected from one or more of the following: an X-ray powder diffraction pattern substantially as depicted in FIG. 4; an X-ray powder diffraction pattern having peaks at 4.6, 9.2, 12.1, 13.9 and 18.5 degrees 2-theta±0.2 degrees 2-theta; and combinations of these data.

Crystalline Form F1 of Elenestinib:Fumaric acid may be further characterized by an X-ray powder diffraction pattern having peaks at 4.6, 9.2, 12.1, 13.9 and 18.5 degrees 2-theta 0.2 degrees 2-theta, and also having any one, two, three, four or five additional peaks selected from 17.1, 18.0, 21.1, 24.5 and 25.3±0.2 degrees 2-theta±0.2 degrees 2-theta.

Crystalline Form F1 of Elenestinib:Fumaric acid may be alternatively characterized by an X-ray powder diffraction pattern having peaks at 4.6, 9.2, 12.1, 13.9, 17.1, 18.0, 18.5, 21.1, 24.5 and 25.3 degrees 2-theta±0.2 degrees 2-theta.

In any aspect or embodiment of the present disclosure, crystalline Form F1 of Elenestinib:Fumaric acid may be isolated. In an embodiments the molar ratio between Elenestinib and the Fumaric acid is between 1:2 and 2:1, preferably between 1:1.

In any aspect or embodiment crystalline form F1 of Elenestinib:Fumaric acid may be chemically pure.

In any aspect or embodiment crystalline Form F1 of Elenestinib:Fumaric acid may be polymorphically pure.

Crystalline Form F1 of Elenestinib:Fumaric acid may be characterized by each of the above characteristics alone/or by all possible combinations.

The present disclosure further encompasses crystalline Elenestinib:Lactic acid. Crystalline Elenestinib:Lactic acid may be a co-crystal of Elenestinib and Lactic acid. Alternatively, crystalline Elenestinib and Lactic acid may be a salt, i.e., Elenestinib Lactate.

As used herein, “Elenestinib:Lactic acid” refers to a salt of Elenestinib and Lactic acid or other complexes (co-crystals) including Elenestinib and Lactic acid wherein the lactic acid is L-lactic acid, D-lactic acid or mixtures thereof (including racemic mixtures i.e., D,L-lactic acid). Preferably, Elenestinib:Lactic acid refers to a salt of Elenestinib and Lactic acid or complex (co-crystal) including Elenestinib and Lactic acid, wherein the Lactic acid is L-lactic acid. According to any aspect or embodiment of the present disclosure, the molar ratio of Elenestinib to Lactic acid (preferably L-lactic acid) is from about 1:2 to about 2:1 preferably about 1:1. Preferably Elenestinib:L-Lactic acid is a salt.

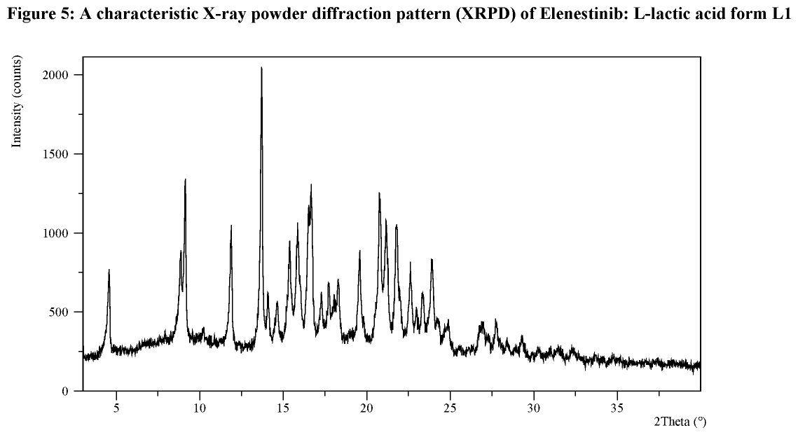

The disclosure further encompasses a crystalline form of Elenestinib:L-lactic acid, designated form L1. Crystalline Form L1 of Elenestinib:L-Lactic acid may be characterized by data selected from one or more of the following: an X-ray powder diffraction pattern substantially as depicted in FIG. 5; an X-ray powder diffraction pattern having peaks at 9.1, 11.9, 13.7, 19.6 and 20.8 degrees 2-theta±0.2 degrees 2-theta; and combinations of these data.

Crystalline Form L1 of Elenestinib:L-lactic acid may be further characterized by an X-ray powder diffraction pattern having peaks at 9.1, 11.9, 13.7, 19.6 and 20.8 degrees 2-theta 0.2 degrees 2-theta, and also having any one, two, three, four or five additional peaks selected from 4.6, 15.9, 21.8, 22.6 and 23.9 degrees 2-theta±0.2 degrees 2-theta.

Crystalline Form L1 of Elenestinib:L-lactic acid may be alternatively characterized by an X-ray powder diffraction pattern having peaks at 4.6, 9.1, 11.9, 13.7, 15.9, 19.6, 20.8, 21.8, 22.6 and 23.9 degrees 2-theta±0.2 degrees 2-theta.

In any aspect or embodiment of the present disclosure, crystalline Form L1 of Elenestinib:L-lactic acid may be isolated.

In any aspect or embodiment crystalline form L1 of Elenestinib:L-lactic acid may be chemically pure.

In any aspect or embodiment crystalline Form L1 of Elenestinib:L-lactic acid may be polymorphically pure.

Crystalline Form L1 of Elenestinib:L-lactic acid may be characterized by each of the above characteristics alone/or by all possible combinations.

As used herein, crystalline Elenestinib:Gluconic acid is a distinct molecular species. Crystalline Elenestinib:Gluconic acid may be a co-crystal of Elenestinib and Gluconic acid. Alternatively crystalline Elenestinib:Gluconic acid may be a salt, i.e., Elenestinib Gluconate.

In an embodiments the molar ratio between Elenestinib and the Gluconic acid is between 1:2 and 2:1, preferably between 1:1.

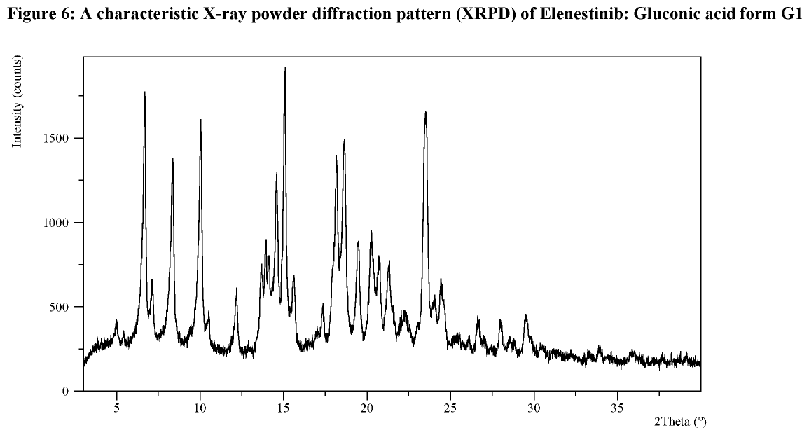

The disclosure further encompasses a crystalline form of Elenestinib:Gluconic acid, designated form G1. Crystalline form G1 of Elenestinib:Gluconic acid may be characterized by data selected from one or more of the following: an X-ray powder diffraction pattern substantially as depicted in FIG. 6; an X-ray powder diffraction pattern having peaks at 6.7, 8.4, 10.1, 15.1 and 23.5 degrees 2-theta±0.2 degrees 2-theta; and combinations of these data.

Crystalline Form G1 of Elenestinib:Gluconic acid may be further characterized by an X-ray powder diffraction pattern having peaks at 6.7, 8.4, 10.1, 15.1 and 23.5 degrees 2-theta±0.2 degrees 2-theta, and also having any one, two, three, four or five additional peaks selected from 12.2, 14.6, 18.2, 18.7 and 19.5 degrees 2-theta±0.2 degrees 2-theta.

Crystalline Form G1 of Elenestinib:Gluconic acid may be alternatively characterized by an X-ray powder diffraction pattern having peaks at 6.7, 8.4, 10.1, 12.2, 14.6, 15.1, 18.2, 18.7, 19.5 and 23.5 degrees 2-theta±0.2 degrees 2-theta.

In any aspect or embodiment of the present disclosure, crystalline Form G1 of Elenestinib Gluconic acid may be isolated.

In any aspect or embodiment crystalline form G1 of Elenestinib:Gluconic acid may be chemically pure.

In any aspect or embodiment crystalline Form G1 of Elenestinib:Gluconic acid may be polymorphically pure.

Crystalline Form G1 of Elenestinib:Gluconic acid may be characterized by each of the above characteristics alone/or by all possible combinations.

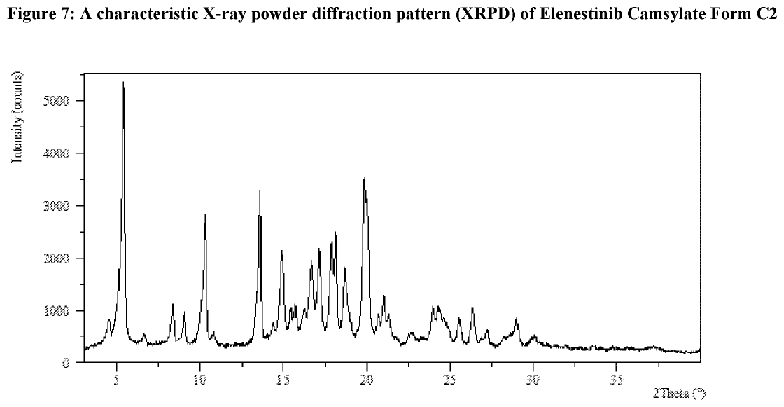

The present disclosure also includes a crystalline polymorph Elenestinib Camsylate designated Form C2. The crystalline Form C2 of Elenestinib Camsylate may be characterized by data selected from one or more of the following: an X-ray powder diffraction pattern substantially as depicted in FIG. 7; an X-ray powder diffraction pattern having peaks at 14.9, 16.7, 17.2, 18.1 and 18.7 degrees 2-theta±0.2 degrees 2-theta; and combinations of these data.

Crystalline Form C2 of Elenestinib Camsylate may be further characterized by an X-ray powder diffraction pattern having peaks at 14.9, 16.7, 17.2, 18.1 and 18.7 degrees 2-theta±0.2 degrees 2-theta, and also having any one, two, three, four or five additional peaks selected from 14.4, 15.7, 17.9, 20.7 and 26.3 degrees 2-theta±0.2 degrees 2-theta.

Crystalline Form C2 of Elenestinib Camsylate may be alternatively characterized by an X-ray powder diffraction pattern having peaks at 14.4, 14.9, 15.7, 16.7, 17.2, 17.9, 18.1, 18.7, 20.7 and 26.3 degrees 2-theta±0.2 degrees 2-theta.

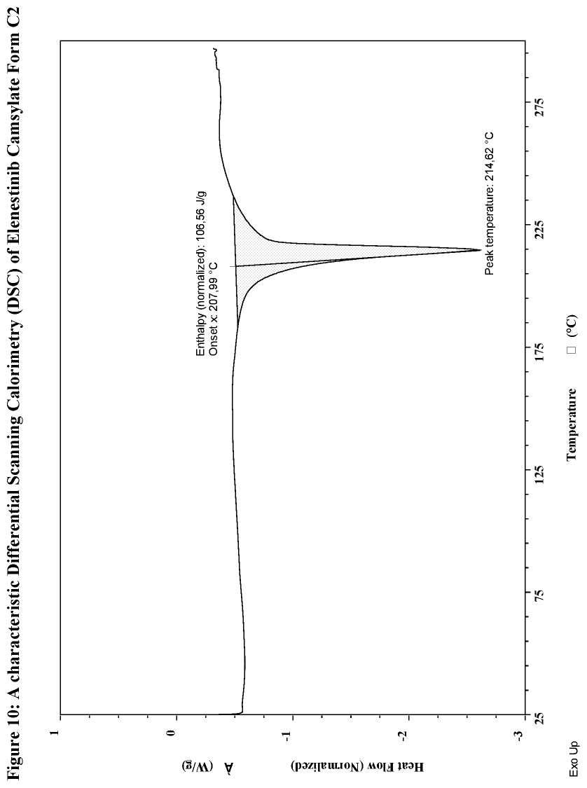

Crystalline Form C2 of Elenestinib Camsylate may be further characterized by an endothermic peak at 214.62 degrees Celsius in DSC thermogram. Crystalline Form C2 of Elenestinib Camsylate may be further characterized by DSC thermogram substantially as depicted in FIG. 10.

In any aspect or embodiment of the present disclosure, crystalline Form C2 of Elenestinib Camsylate may be isolated.

In any aspect or embodiment crystalline form C2 of Elenestinib Camsylate may be chemically pure.

In any aspect or embodiment crystalline Form C2 of Elenestinib Camsylate may be polymorphically pure.

Crystalline Form C2 of Elenestinib Camsylate may be characterized by each of the above characteristics alone/or by all possible combinations.

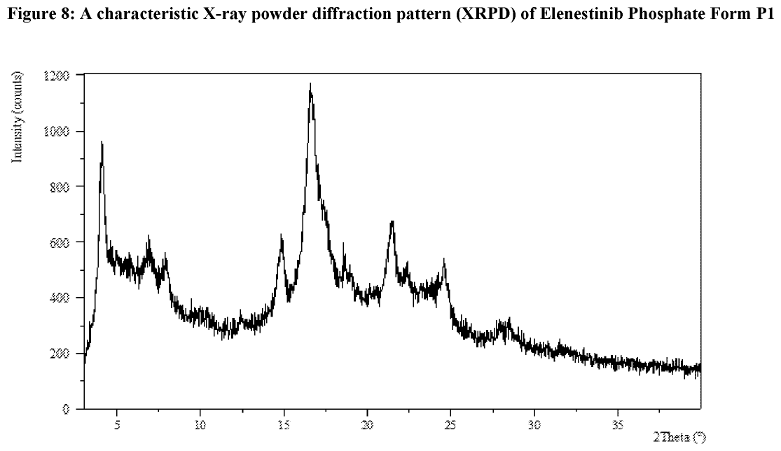

The present disclosure also includes a crystalline polymorph Elenestinib Phosphate designated Form P1. The crystalline Form P1 of Elenestinib Phosphate may be characterized by data selected from one or more of the following: an X-ray powder diffraction pattern substantially as depicted in FIG. 8; an X-ray powder diffraction pattern having peaks at 4.0, 16.4 and 21.5 degrees 2-theta±0.2 degrees 2-theta; and combinations of these data.

Crystalline Form P1 of Elenestinib Phosphate may be further characterized by an X-ray powder diffraction pattern having peaks at 4.0, 16.4 and 21.5 degrees 2-theta±0.2 degrees 2-theta, and also having any one, two, or three additional peaks selected from 8.0, 17.8 and 24.7 degrees 2-theta±0.2 degrees 2-theta.

Crystalline Form P1 of Elenestinib Phosphate may be alternatively characterized by an X-ray powder diffraction pattern having peaks at 4.0, 8.0, 16.4, 17.8, 21.5 and 24.7 degrees 2-theta±0.2 degrees 2-theta.

In any aspect or embodiment of the present disclosure, crystalline Form P1 of Elenestinib Phosphate may be isolated.

In any aspect or embodiment crystalline Form P1 of Elenestinib Phosphate may be chemically pure.

In any aspect or embodiment crystalline Form P1 of Elenestinib Phosphate may be polymorphically pure.

Crystalline Form P1 of Elenestinib Phosphate may be characterized by each of the above characteristics alone/or by all possible combinations.

The present disclosure encompasses a process for preparing solid state forms of Elenestinib salts and solid state forms thereof. The process includes preparing any one of the solid state forms of Elenestinib salts by the processes of the present disclosure, and converting that form to a different salt of Elenestinib or to Elenestinib. The conversion can be done for example by a process comprising basifying any one of the above described forms of Elenestinib salts and reacting the obtained Elenestinib with an appropriate acid, to obtain the corresponding salt. Alternatively, the conversion can be done by salt switching, i.e., reacting any one of the forms of the Elenestinib salts of the present disclosure with an acid having a pKa which is lower than that of the acid of the original salt.

The present disclosure provides the above described salts and crystalline polymorphs of Elenestinib for use in the preparation of pharmaceutical compositions comprising Elenestinib salts and/or solid state forms thereof.

The present disclosure also encompasses the use of salts and crystalline polymorphs of Elenestinib of the present disclosure for the preparation of pharmaceutical compositions of crystalline polymorph Elenestinib salts and/or solid state forms thereof.

The present disclosure includes processes for preparing the above mentioned pharmaceutical compositions. The processes include combining any one or a combination of the salt and crystalline polymorphs of Elenestinib of the present disclosure with at least one pharmaceutically acceptable excipient.

Pharmaceutical combinations or formulations of the present disclosure contain any one or a combination of the solid state form of Elenestinib salts or co-crystals of the present disclosure. In addition to the active ingredient, the pharmaceutical formulations of the present disclosure can contain one or more excipients. Excipients are added to the formulation for a variety of purposes. For example, excipients may be added to assist in formation of formulation suitable for oral administration

Diluents increase the bulk of a solid pharmaceutical composition, and can make a pharmaceutical dosage form containing the composition easier for the patient and caregiver to handle. Diluents for solid compositions include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Elenestinib or salts thereof and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Elenestinib can be administered. For example, it can be administrated orally. Elenestinib may be formulated for administration to a mammal, in embodiments to a human. Elenestinib can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The salts and crystalline polymorph of Elenestinib and the pharmaceutical compositions and/or formulations of Elenestinib of the present disclosure can be used as medicaments, in embodiments for the treatment of systemic mastocytosis

The present disclosure also provides methods of treating of patients with by administering a therapeutically effective amount of any one or a combination of the salts and crystalline polymorphs of Elenestinib of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”)

Sample is powdered in a mortar and pestle and applied directly on a silicon plate holder. The X-ray powder diffraction pattern was measured with Philips X'Pert PRO X-ray powder diffractometer, equipped with Cu irradiation source=1.54184 \hat{Å} (\hat{Å}ngström), X'Celerator (2.022° 2θ) detector. Scanning parameters: angle range: 3-40 deg., step size 0.0167, time per step 37 s, continuous scan.

The described peak positions were determined with or without using silicon powder as an internal standard in an admixture with the sample measured.

### Differential Scanning Calorimetry (DSC)

DSC measurements were done using TA Instruments Discovery, DSC unit. 1-3 mg of sample was weighted in pan, hermetically closed with the pin hole. Sample was purged with 50 ml/min of nitrogen flow and heated in the range of 25-350° C., with heating rate of 10° C./min.

### EXAMPLES

### Preparation of Starting Materials

The starting material Elenestinib Base BF-A-0 can be prepared according to methods known from the literature, for example according to the disclosure of example 10 in International Publication No. WO 2022/192558.

### Purification of Starting Material Elenestinib Base BF-A-0

Unless otherwise indicated the purity is determined by HPLC (area percent). The starting material obtained from example 10 (30 mg) was dissolved in methanol (1.8 ml) at 63° C. Solution was left to cool down to room temperature until crystallization occurred. According to HPLC analysis obtained solid purity was greater than 99.0% (further mentioned as purified BF-A-0). Similarly, Base BF-A-0 can be purified by suspending Base BF-A-0 (3 grams) in 12.6 ml of solvent mixture methanol:water (7% of methanol) at 45° C. for 1 day. After 1 day, suspension was vacuum filtered and washed with 3×2 ml of water to obtain purified BF-A-0.

### Example 1: Preparation of Elenestinib Camsylate Crystal Form C1

A. Purified BF-A-O (48 mg) and 22 mg of camphor-10-sulfonic acid (eq 1.1) were dissolved in 1 ml of solvent system of 2-propanol:water (2:1) at room temperature and mixed for 30 minutes. Solution was put into ice bath and mixing was continued for about 12 hours at 0-5° C. Next, 2 ml of 2-propanol were added, and mixing continued for additional 12 hours at 0-5° C. after which crystallization occurred and suspension was vacuum filtered at room temperature to obtain a solid. The material was analyzed by XRPD and identified as Form C1 as appears in FIG. 1.

B. Purified BF-A-O (67 mg) and 33 mg of camphor-10-sulfonic acid (eq. 1.1) were suspended in 1 ml of 2-propanol at room temperature. After about 24 hours of mixing, suspension was vacuum filtered and analyzed by XRPD to provide the title compound. A similar procedure can also be performed using 1 ml of THF solvent.

### Example 2: Preparation of Elenestinib Succinate Crystal Form S1

Purified BF-A-O (80 mg) and 20 mg of succinic acid (eq 1.1) were suspended in 1 ml of 2-propanol at room temperature. After at least 24 hours of mixing, suspension was vacuum filtered and additionally dried at about 90° C. for 20 minutes. Obtained solid was analyzed by XRPD and Elenestinib Succinate form S1 was identified as appear in FIG. 2.

### Example 3: Preparation of Elenestinib Nicotinate Crystal Form N1

Purified BF-A-O (80 mg) and 20 mg of nicotinic acid (eq 1.1) were suspended in methanol (1 ml) at 55° C. after ~15 minutes, material dissolved and then cool until crystallization occurred at around 45° C. material was vacuum filtered and analyzed by XRPD to obtain form N1 as in FIG. 3.

### Example 4: Preparation of Elenestinib:Fumaric Acid Crystal Form F1

Purified BF-A-O (81 mg) and fumaric acid (19 mg) were suspended in Ethanol (1 ml, 96%) at room temperature. Suspension was mixed for 24 hours then vacuum filtered to obtain a solid material, which was analyzed by XRPD. Form F1 as in FIG. 4 was obtained.

### Example 5: Preparation of Elenestinib:L-Lactic Acid Crystal Form L1

Purified BF-A-O (199 mg) and L-lactic acid (65 μL) were suspended in 2-propanol (3 ml) at 45° C. and mixed for additional one hour, then temperature was set up at 60° C. and suspension was additionally mixed at that temperature for 4 days. Obtained solid was vacuum filtered and analyzed by XRPD. Form L1 was obtained (FIG. 5).

### Example 6: Preparation of Elenestinib:Gluconic Acid Crystal Form G1

Purified BF-A-O (142 mg) and gluconic acid (127.4 mg) were suspended in 2-propanol (3 ml) at 45° C., obtained suspension was mixed for one hour and then temperature was set up at 60° C. Suspension was mixed at 60° C. for 4 days and vacuum filtered. Obtained solid was analyzed by XRPD. Form G1 was obtained (FIG. 6).

### Example 7: Preparation of Elenestinib Camsylate Form C2

1060 mg of Elenestinib base Form A-0 and 940 mg of camphor-10-sulfonic acid were dissolved in 8 ml of solvent mixture methanol/water (ratio 3:1) at 27° C. After 15 minutes of mixing at room temperature crystallization occurred. The suspension was put into an ice bath and mixed for one day. After 24 hours of mixing at 0-5° C., the suspension was vacuum filtered and analyzed by XRPD. Form C2 was obtained. Sample was vacuum dried at 80° C. for 6 hours to remove residual solvents and analyzed by XRPD. According to XRPD analysis Elenestinib Camsylate Form C2 was obtained (FIG. 7).

### Example 8: Preparation of Elenestinib Phosphate Form P1

498 mg of Elenestinib base Form A-0 was suspended in 3.32 ml of methanol at room temperature. 78 μL of phosphoric acid (concentration 85%) was added to the suspension. The suspension was dissolved at room temperature and after 15 minutes of mixing crystallization occurred. 1.66 ml of methanol was added for better mixing. After 2 hours of mixing, the solution was vacuum filtered and analyzed by XRPD. Elenestinib phosphate Form P1 was obtained (FIG. 8).

1. A crystalline form of Elenestinib camsylate.

2. (canceled)

3. A crystalline Form C1 of Elenestinib camsylate according to claim 1, which is characterized by data selected from one or more of the following:
i. an XRPD pattern having peaks at 5.3, 10.2, 13.6, 18.1 and 20.0 degrees 2-theta±0.2 degrees 2-theta; or
ii. an XRPD pattern as depicted in FIG. 1.

4. The crystalline Form C1 of Elenestinib camsylate according to claim 3, which is characterized by an XRPD pattern having peaks at 5.3, 10.2, 13.6, 18.1 and 20.0 degrees 2-theta±0.2 degrees 2-theta, and also having one, two, three, four or five additional peaks selected from 8.3, 16.0, 16.8, 17.3 and 19.4 degrees 2-theta±0.2 degrees 2-theta.

5. A crystalline Form C2 of Elenestinib camsylate according to claim 1, which is characterized by data selected from one or more of the following:
i. an XRPD pattern having peaks at 14.9, 16.7, 17.2, 18.1 and 18.7 degrees 2-theta±0.2 degrees 2-theta; or
ii. an XRPD pattern as depicted in FIG. 7.

6. The crystalline Form C2 of Elenestinib camsylate according to claim 5, which is characterized by an XRPD pattern having peaks at 14.9, 16.7, 17.2, 18.1 and 18.7 degrees 2-theta±0.2 degrees 2-theta, and also having one, two, three, four or five additional peaks selected from 14.4, 15.7, 17.9, 20.7 and 26.3 degrees 2-theta±0.2 degrees 2-theta.

7. A crystalline Form S1 of Elenestinib succinate, which is characterized by data selected from one or more of the following:
i. an XRPD pattern having peaks at 4.6, 11.9, 13.7, 19.6 and 20.9 degrees 2-theta±0.2 degrees 2-theta; or
ii. an XRPD pattern as depicted in FIG. 2.

8. The crystalline Form S1 of Elenestinib succinate according to claim 7, which is characterized by an XRPD pattern having peaks at 4.6, 11.9, 13.7, 19.6 and 20.9 degrees 2-theta±0.2 degrees 2-theta, and also having one, two, three, four or five additional peaks selected from 15.5, 16.8, 17.7, 21.9 and 24.1 degrees 2-theta±0.2 degrees 2-theta.

9. A crystalline Form F1 of Elenestinib:fumaric acid, which is characterized by data selected from one or more of the following:
i. an XRPD pattern having peaks at 4.6, 9.2, 12.1, 13.9 and 18.5 degrees 2-theta±0.2 degrees 2-theta; or
ii. an XRPD pattern as depicted in FIG. 4.

10. The crystalline Form F1 of Elenestinib:fumaric acid according to claim 9, which is characterized by an XRPD pattern having peaks at 4.6, 9.2, 12.1, 13.9 and 18.5 degrees 2-theta±0.2 degrees 2-theta, and also having one, two, three, four or five additional peaks selected from 17.1, 18.0, 21.1, 24.5 and 25.3 degrees 2-theta±0.2 degrees 2-theta.

11. A crystalline Form P1 of Elenestinib phosphate, which is characterized by data selected from one or more of the following:
i. an XRPD pattern having peaks at 4.0, 16.4 and 21.5 degrees 2-theta±0.2 degrees 2-theta; or
ii. an XRPD pattern as depicted in FIG. 8.

12. The crystalline Form P1 of Elenestinib phosphate according to claim 11, which is characterized by an XRPD pattern having peaks at 4.0, 16.4 and 21.5 degrees 2-theta±0.2 degrees 2-theta, and also having one, two, or three additional peaks selected from 8.0, 17.8 and 24.7 degrees 2-theta±0.2 degrees 2-theta.

13. (canceled)

14. (canceled)

15. A pharmaceutical formulation comprising a crystalline Form of Elenestinib camsylate according to claim 1 and at least one pharmaceutically acceptable excipient.

16. A pharmaceutical formulation comprising the crystalline Form C1 of Elenestinib camsylate according to claim 3, and at least one pharmaceutically acceptable excipient.

17. A pharmaceutical formulation comprising the crystalline Form C2 of Elenestinib camsylate according to claim 5, and at least one pharmaceutically acceptable excipient.

18-22. (canceled)

23. A pharmaceutical formulation comprising the crystalline Form S1 of Elenestinib succinate according to claim 7, and at least one pharmaceutically acceptable excipient.

24. A pharmaceutical formulation comprising the crystalline Form F1 of Elenestinib:fumaric acid according to claim 9, and at least one pharmaceutically acceptable excipient.

25. A pharmaceutical formulation comprising the crystalline Form P1 of Elenestinib phosphate according to claim 11, and at least one pharmaceutically acceptable excipient.
